(19) United States
(12) Patent Application Publication
(10) Pub. No.: US 2013/0253061 A1
(43) Pub. Date: Sep. 26, 2013

(54) METHOD OF DROXIDOPA SYNTHESIS

(71) Applicant: CHELSEA THERAPEUTICS, INC., Charlotte, NC (US)

(72) Inventors: Harish K. Pimplaskar, Charlotte, NC (US); Shashikant Vithal Kadam, Pune (IN); Vooradi Mallesh, Zaheerabad Mandal (IN); Pramod Mangaldas Kawale, Jalgaon Maharashtra (IN)

(73) Assignee: CHELSEA THERAPEUTICS, INC., Charlotte, NC (US)

(21) Appl. No.: 13/790,215

(22) Filed: Mar. 8, 2013

Related U.S. Application Data
(60) Provisional application No. 61/613,081, filed on Mar. 20, 2012.

(51) Int. Cl. C07C 227/18; C07C 229/36

(52) U.S. Cl. CPC C07C 227/18; C07C 229/36
USPC 514/567; 562/444

(57) ABSTRACT

The present application relates to a novel method of preparing L-threo-dihydroxyphenylserine (droxidopa). Specifically, the application is directed to a method of preparing droxidopa via a deprotection step that is an alternative to deprotection steps that have been previously disclosed. The new deprotection strategy is advantageous in that it avoids the need to use hydrogenolysis or hydrazine.

### CROSS REFERENCE TO RELATED APPLICATIONS

The present application claims priority to U.S. Provisional Patent Application No. 61/613,081, filed Mar. 20, 2012, the disclosure of which is incorporated herein by reference in its entirety.

### FIELD OF THE INVENTION

The present application is directed to a novel method of preparing L-threo-dihydroxyphenylserine (droxidopa). Specifically, it relates to a method of preparing droxidopa that provides an alternative deprotection strategy to those previously used. The new deprotection strategy avoids the need to use hydrogenation or hydrazine.

### BACKGROUND OF THE INVENTION

L-threo-dihydroxyphenylserine, also known as droxidopa, L-threo-DOPS, or L-DOPS, is an orally active synthetic precursor of norepinephrine. Droxidopa thus replenishes depleted norepinephrine, allowing for re-uptake of norepinephrine into peripheral nervous system neurons. This reuptake, in turn, stimulates receptors for vasoconstriction, providing physiological improvement in symptomatic neurogenic orthostatic hypotension patients. It has also shown efficacy in other diseases, such as Parkinson's disease and depression.

Droxidopa has been used in Japan for many years for the treatment of orthostatic hypotension. It was originally approved in 1989 for the treatment of frozen gait or dizziness associated with Parkinson's disease and for the treatment of orthostatic hypotension, syncope or dizziness associated with Shy-Drager syndrome and Familial Amyloidotic Polyneuropathy. Marketing approval was later expanded to include treatment of vertigo, dizziness and weakness associated with orthostatic hypotension in hemodialysis patients.

The preparation of droxidopa generally involves a multi-step synthesis. Typically, one or more of the necessary steps in the synthesis requires that reactive sites other than that site targeted for reaction are temporarily protected. Thus, the synthesis of droxidopa typically comprises at least one protecting and associated deprotecting step. For example, the catechol moiety, the amine moiety, and/or the carboyxyl moiety may require protection and subsequent deprotection, depending upon the synthetic route and the reagents used in the preparation of droxidopa.

U.S. Pat. Nos. 4,319,040 and 4,480,109 to Ohashi et al. describe processes for the preparation of optically active D- and L-threo-DOPS by optically resolving a racemic mixture of threo-2-(3,4-methylenedioxyphenyl)-N-carbobenzyloxyserine or threo-2-(3,4-dibenzyloxy-phenyl)-N-carbobenzyloxyserine, respectively. Following optical resolution of these racemic mixtures to give the desired L-enantiomer, the methylene or benzyl groups must be removed from the catechol moiety and the carbobenzyloxy (CBZ) group must be removed from the amine group to give droxidopa. The methylene group can be readily removed by reaction with a Lewis acid (e.g., aluminum chloride). The CBZ group (and the benzyl catechol protecting groups, where applicable) is removed from the amine by hydrogenolysis to give the desired compound. The hydrogenolysis step is noted to be carried out by treating the optically resolved salt with hydrogen in the presence of a catalyst, e.g., palladium, platinum, nickel, or the like.

However, for large-scale production of pharmaceutical compounds, hydrogenolysis may not be desirable. For example, hydrogenolysis requires expensive, specialized equipment, which represents a large capital investment. Labor costs are also high, as the process requires careful handling and disposal of certain compounds (e.g., the pyrophoric catalyst). Further, due to the hazards associated with both the reagents and the high pressure system required for hydrogenolysis, it is desirable to avoid synthetic methods that require hydrogenolysis.

In an alternative method for the production of droxidopa, taught by U.S. Pat. No. 4,562,263 to Ohashi et al., hydrogenation is not required. In this process, the amine group is protected via a phthaloyl group. Following optical resolution, the phthaloyl group is removed from the droxidopa precursor by hydrazine. However, hydrazine is known to be genotoxic and has been classified by the EPA as a Group B2 probable human carcinogen. Thus, it is desirable to remove even trace amounts of hydrazine from pharmaceutical compounds. In practice, the method described in the '263 patent suffers from the inability to remove 100% of the hydrazine from the final product. Thus, there is some level of contamination by hydrazine using this method. The Food and Drug Administration has established a maximum genotoxic impurity level of 1.5 micrograms per day. Therefore, based on the maximum daily dose of droxidopa (1.8 g), the maximum allowable hydrazine level therein is 0.8 ppm. Accordingly, it would be advantageous to find a new synthetic route for the preparation of droxidopa that avoids the use of hydrogenolysis and also avoids the use of hydrazine.

### SUMMARY OF THE INVENTION

The present invention provides a novel synthetic route for the preparation of droxidopa. Advantageously, this method does not require the use of hydrazine, thus eliminating the concern of contamination by this reagent in the final product. It also relates to the product and pharmaceutical compositions comprising the product produced according to the method disclosed herein.

In one aspect of the present invention is provided a method for the preparation of droxidopa comprising the step of deprotecting a droxidopa precursor comprising an N-phthaloyl group in the absence of hydrazine to form droxidopa free of residual hydrazine. In certain embodiments, the deprotecting step comprises treating N-phthaloyl-3-(3,4-dihydroxyphenyl)serine with hydroxylamine. The droxidopa may be enantiomerically enriched; for example, the droxidopa is enantomerically enriched for the L-threo isomer. In some embodiments, the L-threo isomer is present at an optical purity of at least about 98%.

In certain embodiments, the synthetic route for the preparation of droxidopa comprises the following steps:

a) converting piperonal to 2-amino-3-(benzo-1,3-diox-5-yl)-3-hydroxypropanoic acid

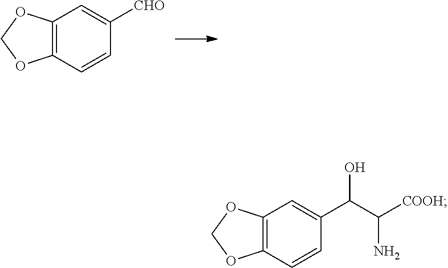 [SMILES: O=Cc1ccc2c(c1)OCO2 ; NC(C(=O)O)C(O)c1ccc2c(c1)OCO2]

b) protecting the free amine

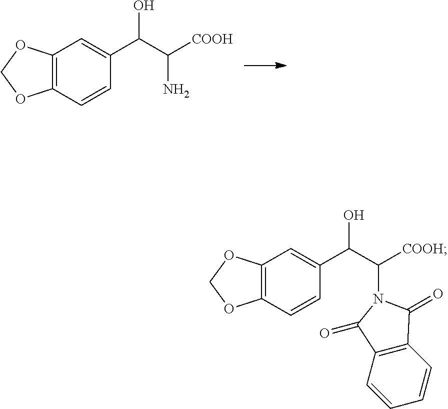 [SMILES: NC(C(=O)O)C(O)c1ccc2c(c1)OCO2 ; O=C(O)C(C(O)c1ccc2c(c1)OCO2)N1C(=O)c2ccccc2C1=O]

c) optical resolution and separation of the desired isomer

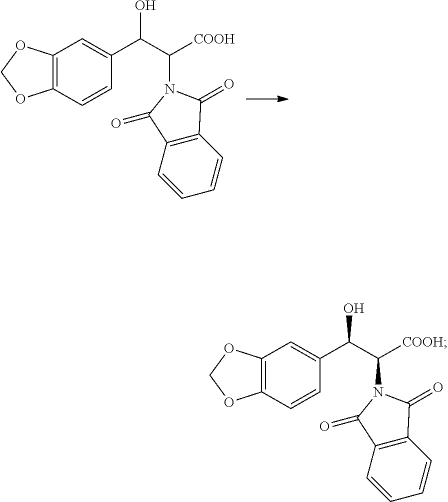 [SMILES: O=C(O)C(C(O)c1ccc2c(c1)OCO2)N1C(=O)c2ccccc2C1=O ; O=C(O)[C@H]([C@H](O)c1ccc2c(c1)OCO2)N1C(=O)c2ccccc2C1=O]

d) removal of the catechol protecting group

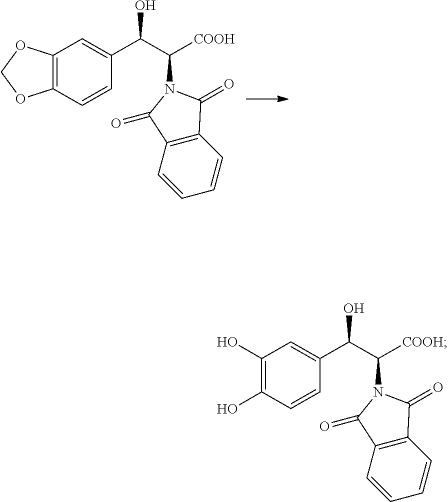 [SMILES: O=C(O)[C@H]([C@H](O)c1ccc2c(c1)OCO2)N1C(=O)c2ccccc2C1=O ; O=C(O)[C@H]([C@H](O)c1ccc(O)c(O)c1)N1C(=O)c2ccccc2C1=O]

and

e) removal of the phthaloyl protecting group

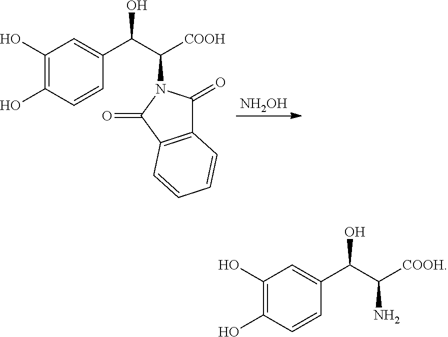

The conditions of the reactions may vary. In certain embodiments, step a) comprises adding glycine in the presence of a base. The base may comprise, for example, sodium hydroxide or potassium hydride. In some embodiments, step a) is conducted in an alcohol solvent, such as methanol or ethanol.

In certain embodiments, step b) comprises adding a phthaloylating agent, selected from the group consisting of phthalic acid, phthaloyl chloride, phthalic anhydride, N-carbomethoxy pthalimide, N-carbethoxy pththalimide, monomethylphthalate, monoethyl phthalate, dimethyl phthalate, diethyl phthalate, and diphenyl phthalate. In some embodiments, the method further comprises the step of reacting phthalimide with ClCOOMe to give N-carbomethoxy phthalimide. In some embodiments, step b) comprises reacting the amine with N-carbomethoxy phthalimide in the presence of Na₂CO₃.

In certain embodiments, step c) comprises adding a chiral derivatizing agent selected from the group consisting of quinidine, quinine, strychnine, cinchonidine, cinchonine, ephedrine, norephedrine, 1-methylamine, dehydroabietylamine, R-2-amino-1,1-diphenyl-1-propanol, S-2-amino-1,1-diphenyl-1-propanol, and L-3-hydroxy-3(4-nitrophenyl)-2-amino-1-propanol. This step may, in some embodiments, further comprise adding an aqueous acidic solution to the product of step c) and extracting the desired isomer with an organic solvent. In one particular embodiment, step c) comprises adding norephedrine in methanol to form an amine salt.

In some embodiments, step d) comprises adding a Lewis acid. The Lewis acid may be selected, for example, from the group consisting of aluminum chloride, aluminum bromide, ferric chloride, stannic chloride, boron trichloride, and boron tribromide. In addition, in certain embodiments, this step further comprises adding a mercaptan of 1-20 carbon atoms with the Lewis acid. In certain embodiments, step e) is conducted in a solvent selected from the group consisting of methanol, ethanol, water, and mixtures thereof.

The product may have a relatively high level of optical purity. For example, in some embodiments, the product has an optical purity of greater than about 90%, greater than about 95%, or greater than about 98%. The product may comprise little to no hydrazine. For example, in certain embodiments, the product comprises less than about 0.05% by weight hydrazine, less than about 0.02% by weight hydrazine, less than about 0.01% by weight hydrazine, or 0.0% by weight hydrazine.

In another aspect of the present invention is provided L-threo-dihydroxyphenylserine, produced according to the methods disclosed herein. In another aspect of the invention is provided a composition comprising droxidopa synthesized from an N-phthaloyl protected precursor, wherein the droxidopa is free of residual hydrazine. The composition may, in certain embodiments, be a pharmaceutical composition comprising droxidopa and one or more pharmaceutically acceptable excipients. In some embodiments, the droxidopa in the composition is enantomerically enriched for the L-threo isomer. For example, the L-threo isomer may be present at an optical purity of at least about 98%.

### DETAILED DESCRIPTION OF THE INVENTION

The present invention now will be described more fully hereinafter. However, the invention may be embodied in many different forms and should not be construed as limited to the embodiments set forth herein; rather, these embodiments are provided so that this disclosure will satisfy applicable legal requirements. Like numbers refer to like elements throughout. As used in the specification, and in the appended claims, the singular forms “a”, “an”, “the”, include plural referents unless the context clearly dictates otherwise.

The present invention provides a novel process for the production of droxidopa. Specifically, the invention provides a process for preparing droxidopa which avoids using hydrogenation and/or hydrazine. Further, the invention may provide a process that simplifies and increases the efficiency of preparing droxidopa. Advantageously, in certain embodiments, the method provides for a reduction in the number of filtration and pH adjustment steps that are commonly involved in other methods for the synthesis of droxidopa.

The chemical structure of dihydroxyphenylserine is provided in Formula I, below. Due to the presence of two chiral carbon atoms in the compound, there are four possible stereoisomers of the compound. The L-threo-enantiomer, droxidopa, is the only biologically active stereoisomer.

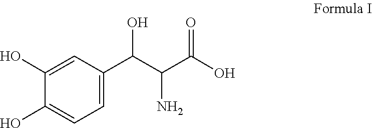 [SMILES: NC(C(=O)O)C(O)c1ccc(O)c(O)c1]

The preparation of this compound generally involves a multi-step synthesis. Due to the presence of various reactive functional groups on the compound, the synthesis of droxidopa typically comprises at least one protecting and associated deprotecting step. For example, the catechol moiety, the amine moiety, and/or the carboxyl moiety on droxidopa precursors may require protection and subsequent deprotection, depending upon the synthetic route and the reagents used in the preparation of droxidopa. A droxidopa precursor is any compound that, upon undergoing one or more chemical reactions, can be converted in some percentage to droxidopa. Depending on the stage of the reaction, the droxidopa precursor can comprise functional groups that are unprotected, partially protected, or fully protected. For example, certain droxidopa precursors include, but are not limited to, piperonal, 2-amino-3-(benzo-1,3-diox-5-yl)-3-hydroxypropanoic acid, 2-phthalimido-3-hydroxy-3-(3,4-methylenedioxyphenyl)propionic acid, L-threo-(N-phthaloyl-3-(3,4-methylenedioxyphenyl)serine) norephedrine, L-threo (N-phthaloyl-3-(3,4-methylenedioxyphenyl)serine), and L-threo(N-phthaloyl-3-(3,4-dihydroxyphenyl)serine). It is understood that, in addition to these compounds, analogous compounds comprising other functional groups and/or other protecting groups (e.g., other catechol protecting groups) are considered to be droxidopa precursors.

Methods for protecting and deprotecting various functional groups are well known. For exemplary reagents and protocols for the protection and deprotection of a wide range of functional groups, see for example T. W. Green, P. G. M. Wuts, Protective Groups in Organic Synthesis, Wiley-Interscience, New York, 1999, 583-584, 744-747, which is incorporated herein by reference. The protecting groups must be carefully selected such that they fulfill certain criteria. For example, a protecting group should be capable of being introduced selectively at the moiety to be protected, without any adverse effects on other moieties in the molecule. A protecting group should react with the moiety to be protected to give a protected substrate that is stable to the projected subsequent reaction conditions. It should introduce minimal additional functionality to the molecule, to avoid further reaction. A protecting group should also be readily removable in high yield such that the regenerated functional group and the other moieties on the substrate are unaffected.

There are numerous protecting groups for catechols that satisfy the above criteria and can thus be employed in the preparation of droxidopa. As specific examples, the catechol group of droxidopa precursors is commonly protected with tert-butyl diphenyl silane (TBDPS), acetonide, benzyl groups, or is protected as a methylenedioxy moiety or a cyclic ethyl orthoformate moiety. The exemplary reaction schemes provided and discussed herein relate to compounds wherein the catechol is protected as a methylenedioxy group; however, these schemes can be readily adapted for applicability to compounds with other catechol protecting groups. Thus, although protection of the catechol as a methylenedioxy is preferred, the invention is also intended to encompass reactions wherein an alternative catechol protecting group is used.

To protect the amine functionality on droxidopa precursors, fewer protecting groups are available that satisfy the above criteria. As taught by the '040 and '109 patents referenced herein, CBZ can be used to protect the amine. However, effective removal of the CBZ group is typically accomplished by hydrogenolysis, which has been noted previously to be undesirable. The '263 patent referenced herein teaches the use of a phthaloyl group to protect the amine. The phthaloyl group can be introduced by any method. Various phthaloylating agents are known, including, but not limited to, phthalic acid, phthalic acid derivatives (e.g., phthalic anhydride, N-carbomethoxy phthalimide, N-carbethoxy phthalimide, phthaloyl chloride), and phthalic acid esters (such as mono- or di-alkyl or aryl esters, including ethyl phthalate, monomethylphthalate, dimethyl-phthalate, diethyl phthalate, diphenyl phthalate and dimethyl phthalate).

According to the present invention, a method of preparation of droxidopa is provided, wherein the phthaloyl protecting group is removed by hydroxylamine to give a free amine. The inventors have surprisingly found that, although numerous other deprotection strategies have been tested and shown to be unsuccessful, deprotection with hydroxylamine provides a product that is comparable to the products produced via prior art methods as disclosed above, and avoids the use of hydrogenolysis or hydrazine.

In one embodiment, the method of preparation of droxidopa is provided in Scheme 1, below:

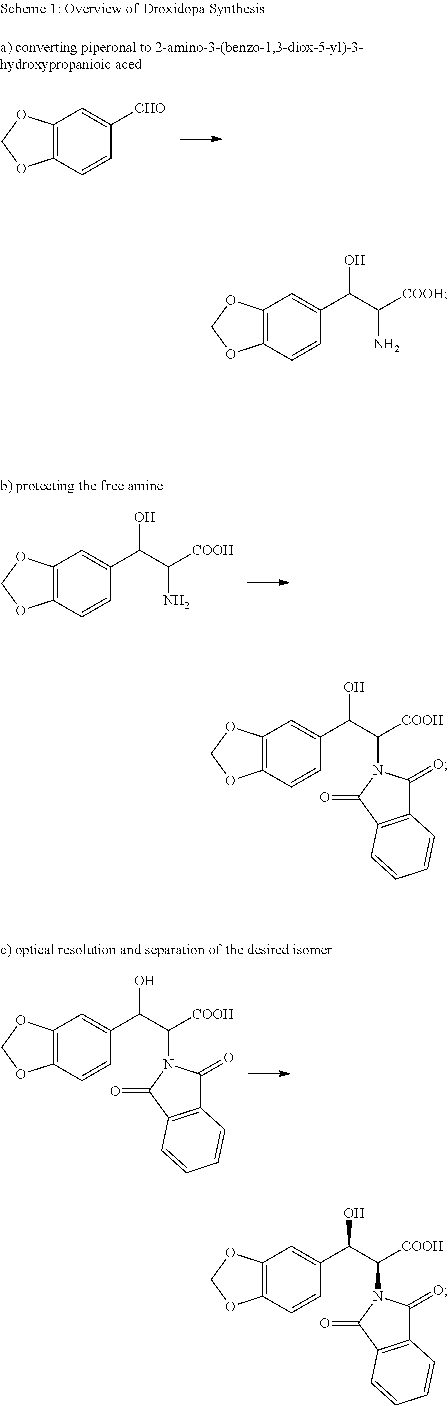 [SMILES: O=Cc1ccc2c(c1)OCO2 ; NC(C(=O)O)C(O)c1ccc2c(c1)OCO2 ; NC(C(=O)O)C(O)c1ccc2c(c1)OCO2 ; O=C(O)C(C(O)c1ccc2c(c1)OCO2)N1C(=O)c2ccccc2C1=O ; O=C(O)C(C(O)c1ccc2c(c1)OCO2)N1C(=O)c2ccccc2C1=O ; O=C(O)[C@H]([C@H](O)c1ccc2c(c1)OCO2)N1C(=O)c2ccccc2C1=O]

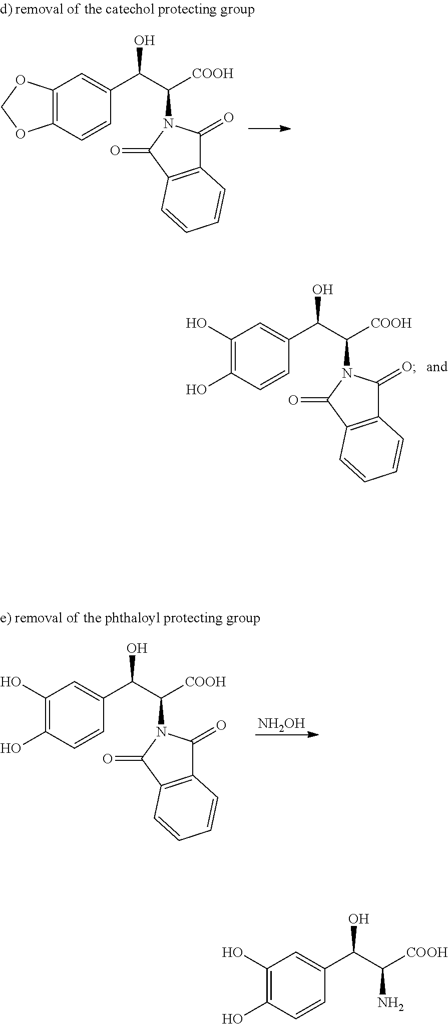

The reagents, solvents, and conditions of the above reaction steps can vary. One of skill in the art would be aware that reagents, solvents, and conditions can often be modified, e.g., to provide the desired product in a better yield or higher purity or to avoid the use of certain reagents. Exemplary reaction conditions are provided, and certain variations thereof are intended to be encompassed by the present invention.

Step a) is the preparation of 3-(3,4-methylenedioxyphenyl)serine (i.e., 2-amino-3-(benzo-1,3-dioxol-5-yl)-3-hydroxypropanoic acid). It is typically conducted by combining 1,3-benzodioxole-5-carbaldehyde (piperonal) with glycine in the presence of a base (e.g., sodium hydroxide or potassium hydride). The solvent can be any suitable solvent, and is typically an alcohol (e.g., methanol or ethanol), toluene, or a mixture thereof. The reaction temperature can vary and is generally from about 50-70° C. The ratio of reagents can vary and in some embodiments, the ratio is about 2:1:2 piperonal:glycine:base. Following reaction, the mixture is generally treated with acid (e.g., acetic acid or HCl). During the workup of the reaction, it may be necessary to further adjust the pH of the solution, which can be done using any acid and/or base, e.g., HCl, glacial acetic acid, and/or NaOH. In certain embodiments, the transformation of Step a) is effected using glycine and KOH in methanol or toluene, followed by treatment with dilute HCl to give 3-(3,4-methylenedioxyphenyl)serine.

Step b) is the protection of the free amine with a phthaloyl protecting group, giving 2-phthalimido-3-hydroxy-3-(3,4-methylenedioxyphenyl)propanoic acid. This step can be accomplished with various types of phthaloylating agents. For example, phthaloylating agents include phthalic acid, phthalic acid derivatives (e.g., phthalic anhydride, N-carbomethoxy phthalimide, N-carbethoxy phthalimide, phthaloyl chloride), and esters of phthalic acid (such as mono- or di-alkyl or aryl esters, including monoethyl phthalate, monomethyl phthalate, dimethyl phthalate, diethyl phthalate, and diphenyl phthalate).

In certain embodiments, N-carbomethoxy phthalimide is used as a phthaloylating agent. N-carbomethoxy phthalimide can, in certain embodiments, be prepared via the conversion of phthalimide, as shown below in Scheme 2.

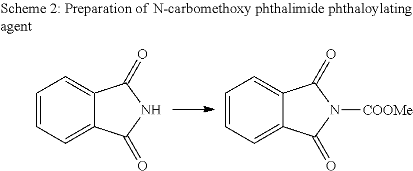 [SMILES: O=C1NC(=O)c2ccccc21 ; COC(=O)N1C(=O)c2ccccc2C1=O]

This conversion of phthalimide to N-carbomethoxy phthalimide can, in some embodiments, be effected by reacting the phthalimide with methyl chloroformate (ClCOOMe) and triethylamine in DMF. The temperature at which the reaction is conducted is typically relatively low, e.g., from about 0-5° C. Upon quenching the reaction (e.g., with water) and drying the product, N-carbomethoxy phthalimide is provided.

In certain embodiments, the free amine of 3-(3,4-methylenedioxyphenyl)serine can be reacted with the N-carbomethoxy phthalimide and Na₂CO₃ in water. The reaction can be conducted, for example, at a temperature of about 30° C. In some embodiments, the pH of the solution must be adjusted following reaction to give an acidic solution (e.g., with dilute sulfuric acid), from which the phthaloyl-protected amine, namely, 2-phthalimido-3-hydroxy-3-(3,4-methylenedioxyphenyl)propanoic acid, can be obtained.

Step c) is the chiral resolution of the compound into its two enantiomeric forms, followed be separation of the desired enantiomer. Optical resolution is any method by which a mixture is separated into its enantiomeric components. Typically, this is done by derivatization of the compounds with optically pure reagents (derivatizing agents), which results in the formation of a pair of diastereomers. According to the invention, any derivatizing agent can be used which can react with the compound, resulting in the formation of diastereomers from the mixture of enantiomers. Exemplary derivatizing agents include, but are not limited to, optically active amines such as quinidine, quinine, strychnine, cinchonidine, cinchonine, ephedrine, norephedrine, 1-methylamine, dehydroabietylamine, R-2-amino-1,1-diphenyl-1-propanol, S-2-amino-1,1-diphenyl-1-propanol, and L-3-hydroxy-3(4-nitrophenyl)-2-amino-1-propanol. The ratio of derivatizing agent to racemic compound is typically about 0.5:1 to 1:1. This derivatization can be done in any suitable solvent. For example, suitable solvents include, but are not limited to, alcohols (e.g., methanol, ethanol, and isopropanol), ethers (e.g., tetrahydrofuran and dioxane), acetonitrile, water, and mixtures thereof.

The resulting diastereomeric salts can then be separated by conventional techniques. For example, recrystallization can be used. In certain embodiments, an aqueous acidic solution (e.g., hydrochloric acid, sulfuric acid, or phosphoric acid) is added to protonate the salt. The acid is typically added in an amount of at least about 1 mole per mole of salt. The desired isomer can then be extracted with an organic solvent (e.g., ethyl acetate, chloroform, dichloroethane, dichloromethane, diethyl ether, and combinations thereof).

In one exemplary embodiment, the product of step b) is treated with norephedrine in methanol to form an amine salt. In such embodiments, the resulting salt is generally protonated with an acidic solution, such as a 40% aqueous H₂SO₄ solution, to give L-threo(N-phthaloyl-3-(3,4-methylenedioxyphenyl)serine).

Step d) is the removal of the protecting group from the catechol moiety to give L-threo(N-phtyaloyl-3-(3,4-dihydroxyphenyl)serine). Various methods can be used to remove the catechol protecting group and can be used according to the invention. The illustrated protecting group can be removed, for example, by treating the compound with a Lewis acid in a suitable solvent. Exemplary Lewis acids that can be used for this purpose include, but are not limited to, aluminum chloride, aluminum bromide, ferric chloride, stannic chloride, boron trichloride, and boron tribromide. Alternatively, a complex of a Lewis acid and dimethyl sulfide can also be used. The amount of Lewis acid used can vary, and can be, for example, in a ratio of about 1:20, preferably 2:10 moles Lewis acid: moles (2S,3R)-3-(benzo[d][1,3]dioxol-5-yl)-2-(1,3-dioxoisoindolin-2-yl)-3-hydroxypropanoic acid. In certain embodiments, a mercaptan of 1-20 carbon atoms can be added (e.g., methylmercaptan, ethylmercaptan, butylmercaptan, octylmercaptan, dodecanylmercaptan, octadecanylmercaptan, thiophenol, and mixture thereof). Where used, the mercaptan additive can be used in an amount of about 1 to 5 moles per mole of Lewis acid. This step can be conducted in any suitable solvent. Preferable solvents include halogenated hydrocarbons (e.g., dichloromethane, chloroform, dichloroethane, and chlorobenzene), aromatic hydrocarbons (e.g., toluene and benzene), esters (e.g., ethyl acetate and butyl acetate), nitrohydrocarbons (e.g., nitromethane, nitroethane, nitrobenzene), ketones (e.g., acetone, methyl ethyl ketone), pyridine, and mixtures thereof. Although the reaction can be conducted at any temperature, it is preferably done in the range of about −40° C. to about 80° C. (e.g., −10° C. to 30° C.). The isolation of the L-threo-(N-phthaloyl-3-(3,4-dihydroxyphenyl)serine may further involve one or more pH adjustment steps (e.g., with oxalic acid) and/or one or more recrystallization steps. In one specific embodiment, this deprotection is done using aluminum chloride and octylmercaptan in chlorobenzene or dichloromethane.

Step e) is the removal of the phthaloyl protecting group to give the free amine and to provide the final product. According to the invention, the protecting group is removed via NH₂OH (hydroxylamine). The hydroxylamine can be provided, in certain embodiments, in the form of an aqueous solution. In some embodiments, the hydroxylamine can be provided as hydroxylamine HCl (i.e., hydroxylammonium chloride). In certain embodiments, NaHCO₃ is added to the reaction mixture. The phthaloyl deprotection can be accomplished in any suitable solvent. For example, solvents that can be used for this phthaloyl deprotection include water, alcohols (e.g., methanol, ethanol, or isopropyl alcohol), and mixtures thereof. In some embodiments, the reaction mixture is refluxed to provide the desired product.

Purification of the desired product can be accomplished in various ways. For example, in certain embodiments, a hydrochloride salt is first formed, which is insoluble in water and/or isopropyl alcohol and can thus be isolated from a solution of these solvents, which results in the removal of certain impurities. This step can be repeated if necessary to reduce the level of impurities below a desired level (e.g., less than or equal to about 0.05% impurity). The resulting compound can be further purified. For example, the compound can be dissolved in aqueous HCl and combined with activated carbon and/or celite. The purified compound is converted to L-threo(3,4-dihydroxyphenyl)serine, for example, through treatment with base. In one embodiment, the compound is combined with triethylamine in methanol and dried to give droxidopa.

Although the use of protecting groups is generally well known in organic chemistry, the particular reagents that work for a given system are often not known in advance. Surprisingly, the inventors have found that, with regard to the present invention, numerous reagents that are commonly used to remove phthaloyl protecting groups were unsuccessful in the reaction scheme described above. Table 1, below, provides a summary of trials with various reagents for the removal of the phthaloyl protecting group.

Hydroxylamine typically removes the phthaloyl protecting group with greater than about 90% efficiency, preferably greater than about 95% efficiency, and more preferably greater than about 99% efficiency. In certain embodiments, the hydroxylamine removes the phthaloyl protecting group with greater than 99.9% efficiency, including 100% efficiency. In preferred embodiments, the hydroxylamine reagent is completely removed from the final product. For example, the final product (droxidopa) produced according to the present invention preferably comprises less than about 0.05% by weight, preferably less than about 0.02% by weight, more preferably less than about 0.01% by weight, and most preferably 0% by weight residual hydroxylamine.

Advantageously, the process of the present invention does not use hydrazine to produce droxidopa. Therefore, the invention provides droxidopa having less than about 0.05% by weight hydrazine, less than about 0.02% by weight hydrazine, or less than about 0.01% by weight hydrazine. Typically, the present invention can provide a product comprising 0.0% by weight hydrazine.

The final product preferably has a high optical purity. Optical purity can be measured by any means. For example, polarimetry, chiral chromatography, and/or NMR spectroscopy can be used to measure and/or calculate optical purity. For example, the optical purity of droxidopa produced according to the present invention is typically greater than about 80%, preferably greater than about 90%, more preferably greater than about 95%, and most preferably greater than about 98%. In certain embodiments, the optical purity is greater than about 99%, including 100%. Optical purities as provided herein advantageously refer to the L-threo isomer.

In another aspect, the invention provides a composition comprising droxidopa prepared according to the methods described in the present application. For example, in certain embodiments, a pharmaceutical composition is provided, comprising droxidopa prepared according to the methods disclosed herein in combination with one or more pharmaceutically acceptable excipients. Droxidopa produced according to the methods disclosed herein and pharmaceutical compositions thereof can be used for the treatment of any condition that may be responsive to the administration of droxidopa. Representative pharmaceutical compositions and disorders for which droxidopa or compositions thereof may be used can vary, and may include, for example, those compositions and disorders described in U.S. Patent Application Publication Nos. 2008/0015181, 2008/0221170, 2008/022,7830, and 2009/0023705, all to Roberts et al., which are incorporated herein by reference in their entireties.

### EXPERIMENTAL SECTION

### Example 1

### Screening of Deprotection Strategies for Phthaloyl Group

TABLE 1
Comparison of deprotecting trial reactions

| Reaction Conditions | Results | Reference |
|---|---|---|
| Ethylene diamine (excess), | Starting material was | Carbohydrate Res. 234: 139 |
| n-BuOH, 70° C. | consumed, but no product was | (1993) |
| | detected | |
| Ethylene diamine (excess), | Starting material was | Carbohydrate Res. 234: 139 |
| n-BuOH, 90° C. | consumed, but no product was | (1993) |
| | detected | |
| 40% aq. MeNH₂, | Starting material was | U.S. Patent application Pub. No. |
| water, 60° C. | consumed, but no product was | 2005/0250949 to Albizati |
| | detected | et al. |
| 5 eq. BuNH₂, MeOH, | Unreacted starting material | Tetrahedron Lett. 20(42): |
| reflux, 16 h | | 4013-4016 (1979) |
| 6M HCl, AcOH, reflux 16 h | Starting material was | Tetrahedron: Asymmetry |
| | consumed, but no product was | 10(3): 493-509 (1999) |
| | detected | |
| NaBH4/AcOH, IPA/water, | Starting material was | J. Carbohydrate Chem. |
| 70° C. | consumed, but no product was | 7(3): 701 (1988) |
| | detected | |
| 1.5 eq. NH₂OH, | Clean product by ¹H NMR | |
| MeOH/water, 60-65° C. | | |

### Example 2

### Exemplary Synthesis of Droxidopa

The synthesis of droxidopa according to the methods provided herein can be conducted as a continuous process or can be conducted in a series of individual steps. Both processes are intended to be encompassed by the present disclosure.

### Synthesis of N-carbomethoxy phthalimide

| Raw Material | Quantity |
|---|---|
| Phthalimide | 120.0 kg |
| Dimethylformamide (DMF) | 420 ± 10 L |
| Triethylamine (TEA) | 124.0 L ± SQ |
| Methylchloroformate (MCF) | 85.0 kg ± SQ |
| Demineralized Water | 2340 ± 40 L |

3-Methoxy phthalimide 1 (120 kg) is added to a vessel containing dimethylformamide (420 L) and stirred (95±10 RPM) at 25-30° C. for 30 min. The contents are cooled to 18-20° C. and triethylamine (124 L) is added. The contents are further cooled to −10° C. to −5° C. and methylchloroformate 2 (85 kg) is added. The reaction temperature is maintained in the range of −10° C. to 0° C. to control the exothermicity during the addition of methylchloroformate. The temperature of the mixture is maintained at 0-5° C. for 1 h after the addition of methylchloroformate.

The reaction mixture is then heated to 25-30° C. for 1 h. An in-process sample is taken to confirm a phthalimide content limit ≦2.5%. The mixture is sampled again to confirm a phthalimide content ≦0.5%. The mixture is transferred to another reactor, cooled to 0-5° C., and the reaction is quenched with the addition of demineralized water (1260±10 L) at a temperature of 10±5° C. The mixture is then heated at 25-30° C. for 1 h.

The material is centrifuged for 2 h and the wet cake is washed three times with demineralized water (360 L). The wet cake is dried at a temperature of 55-60° C. and a sample is taken after 12 h of drying to confirm water content ≦1.0% w/w.

Expected yield of N-carbomethoxy phthalimide (3): 144-158 kg. This material is not isolated and is used directly in the next step.

### Synthesis of 2-amino-3-(benzo-1,3-dioxol-5-yl)-3-hydroxypropanoic acid

| Raw Material | Standard Quantity |
|---|---|
| Potassium Hydroxide | 85.0 ± 1 kg |
| Methanol | 415 ± 5 L |
| Glycine | 52 ± 1 kg |
| Toluene | 1585 ± 35 L + SQ |
| Piperonal | 229 ± 1 kg |
| Hydrochloric Acid | 230 ± 5 kg |
| Glacial Acetic Acid | 218 ± 2 kg |
| Caustic Flakes | 200 ± 6 kg |
| Demineralized Water | 1870 ± 35 L |

Piperonal 4 (229±1 kg) is added to toluene (310±5 L) in a reactor and the mixture is stirred (85-95 RPM) until a clear solution is obtained (approximately 30 min). The piperonal solution is transferred to a vessel for later use. Methanol (415±5 L) is added to the reactor followed by the addition of potassium hydroxide (85 kg). The mixture is stirred for approximately 30 min at 25-30° C. to provide a clear solution. The potassium hydroxide solution is cooled to 20-25° C., and then glycine 5 (52±1 kg) and toluene (310±5 L) are added while stirring at 20-25° C. The contents of the reactor are cooled to 15-20° C. The solution of piperonal in toluene is slowly added to the reactor while maintaining the temperature at 15-20° C. The reactor temperature is increased to 20-25° C. and maintained for 18 h. An in-process sample is taken to determine glycine content by TLC (limit ≦5.0%).

The reaction mass is transferred to another reactor, the temperature is increased to 40° C., and the solvents (toluene and methanol) are distilled off under vacuum until the mixture becomes thick. Additional toluene (210±5 L) is added to the reaction mass three times and distilled out for complete removal of methanol and toluene. The reaction mixture is kept under vacuum at 40° C. After 3 h, the reaction mixture is cooled to 18-22° C. and a dilute hydrochloric acid solution (230±5 L hydrochloric acid and 1145±10 L demineralized water) is added and mixed for 30 min.

The mixture is allowed to settle for 30 min to separate into organic and aqueous layers. The aqueous layer is washed with toluene (310±5 L) and separated. Glacial acetic acid (218±2 kg) is added to the washed aqueous layer at 20-25° C. Caustic solution (580±5 L DM Water and 200±1 kg caustic flakes) is slowly added into the reaction mass to bring the pH 5.0 to 5.1 while maintaining the temperature at 25-30° C. The pH of the mixture is brought to 5.45-5.50 at 25-30° C., while stirring for 30 min. The mixture is centrifuged for 8 h 30 min to 9 h and the resulting wet cake is washed with demineralized water (50±5 L). The cake is dried at 50-55° C. under vacuum, and a sample is taken after 12 h to confirm that water content is ≦10% w/w.

The purity is analyzed by HPLC (limit≦10%).
Expected yield of 2-amino-3-(benzo-1,3-dioxol-5-yl)-3-hydroxypropanoic acid (6): 135-145 kg.

### Synthesis of 2-phthalimido-3-hydroxy-3-(3,4-methylenedioxyphenyl)propionic acid

| Raw Material | Standard Ratio |
|---|---|
| N-carbomethoxy phthalimide (3) | 140 kg |
| 2-amino-3-(benzo-1,3-dioxol-5- | 140 kg |
| yl)-3-hydroxypropanoic acid (6) | |
| Soda Ash | 66 kg |
| Sulfuric Acid | 134 kg |
| Demineralized Water | 1120, 196, 1092, 518, |
| | 518, 2590, 518, and 518 L |

Intermediate 6 (140 kg) is added to a reactor containing demineralized water (1120 L) and stirred (85-95 RPM) for 10 min at 20-25° C. The contents are cooled to 15-20° C. and compound 3 (140 kg) is added followed by a sodium carbonate solution (63.5-68.3 kg sodium carbonate in 189-203 L demineralized water) within 45-60 min. The mixture is heated to 30-35° C. and held for 90 min. An in-process sample is taken to measure for Stage II (≦2.5%) and Stage-I intermediate (≦2.5%). After acceptance criteria are met, the mixture is cooled to 15-20° C. A dilute sulfuric acid solution (134 kg sulfuric acid in 1120 L demineralized water) at 15-20° C. is added to the mixture to bring the pH to 1.0-2.0. The mixture is maintained at this temperature and pH for 30 min, and then the mixture is heated to 20-25° C. for 2 h. The mixture is centrifuged for 9 h and the resulting wet cake is washed twice with 518 L of demineralized water. The wet cake is removed from the centrifuge, washed in a reactor containing demineralized water (2590 L), and stirred for 1 h at 25-30° C. The material is centrifuged for 9 h and the wet cake is washed twice with demineralized water (518 L). The final wet cake is dried at 45-50° C. under vacuum until water content is ≦1.0% w/w. Intermediate (6) output is considered as standard input and a mean of 140 kg is taken for all inputs.

Expected yield of 2-Phthalimido-3-hydroxy-3-(3,4-methylenedioxyphenyl)propionic acid (7): 187-208 kg.

### Synthesis of L-threo (N-phthaloyl-3-(3,4-methylenedioxyphenyl)serine) norephedrine salt

| Raw Material | Standard Ratio | |
|---|---|---|
| 2-phthalimido-3-hydroxy-3-(3,4- | 197.5 | kg |
| methylenedioxyphenyl)propionic acid (7) | | |
| L-Norephedrine | 89 | kg |
| Methanol | 296, 395, 49, 197.5, and 395 L | |
| L-threo(N-phthaloyl-3-(3,4- | 50 | g |
| methylenedioxyphenyl)serine for seeding | | |

L-Norephedrine 8 (89 kg) is added to a reactor containing methanol (296 L) and stirring (45-50 RPM) is started. The mixture is maintained at 25-30° C. for 15-20 min, and then transferred into a vessel for later use.

2-Phthalimido-3-hydroxy-3-(3,4-methylenedioxyphenyl)propionic acid 7 (197.5 kg) is added to a reactor containing methanol (395 L). The material is stirred for 15-20 min at 25-30° C. The L-norephedrine solution is added and mixed for 3 h. If precipitation is not observed within 30 min of adding the L-norephedrine solution, it is seeded with L-threo(N-phthaloyl-3-(3,4-methylenedioxyphenyl)serine (approximately 50 g). After 3 h of mixing, the mixture is cooled to 10-15° C. and maintained for 1 h. An in-process sample is taken to check for purity by HPLC (≧99.0% a/a). The mixture is centrifuged for 1 h to 1 h 30 min and the wet cake is washed with methanol (49 L) followed by isopropyl alcohol (197.5 L). The wet cake is checked for purity. If purity is <99% ala, the wet cake is washed with a prechilled solution of methanol (197.5 L) followed by isopropyl alcohol (99 L). After achieving the required purity level, as measured by HPLC, the wet cake is removed from the centrifuge. The cake is dried at 45-50° C. until loss on drying ≦1.0% w/w.

Expected yield of L-threo (N-phthaloyl-3-(3,4-methylenedioxyphenyl)serine) norephedrine (9) salt: 85-99 kg.

### Synthesis of L-threo (N-phthaloyl-3-(3,4-methylenedioxyphenyl)serine)

| Raw Material | Standard Ratio |
|---|---|
| L-threo (N-phthaloyl-3-(3,4- | 92 kg |
| methylenedioxyphenyl)serine) | |
| norephedrine (9) | |
| Sulfuric Acid (CP) | 20 kg |
| Demineralized Water | 552, 138, 138, 138, 460, 138, and 138 L |

Demineralized water (552 L) is added to a reactor and cooled to 10-15° C. Sulfuric acid (20 kg) is added while maintaining the temperature below 30° C. and stirring for 15-20 min. The solution is cooled to 15-20° C. and 9 (92 kg) is slowly added while stirring and maintaining temperature. The solution is heated to 45-50° C. for 6 h, cooled to 25-30° C., and held for 1 h. The pH is checked to confirm the solution is <2.0.

The mixture is centrifuged for 1 h and the wet cake is washed two times with demineralized water (138 L). The wet cake is removed and added to a reactor containing demineralized water (460 L). The temperature is maintained at 25-30° C. and stirred for 1 h. The material is centrifuged for 30 min and the wet cake is washed two times with demineralized water (138 L). The material is collected and placed into preweighed containers.

Expected yield of L-threo(N-phthaloyl-3-(3,4-methylenedioxyphenyl)serine) (10): 60-64 kg.

### Synthesis of L-threo(N-phthaloyl-3-(3,4-dihydroxyphenyl)serine)

| Raw Material | Standard Ratio |
|---|---|
| L-threo(N-phthaloyl-3-(3,4- | 62 kg |
| methylenedioxyphenyl)serine) (10) | |
| Methylene Chloride | 1240, 1550, 186, and 6.2 L |
| Octanethiol | 78 kg |
| Aluminum Chloride | 81 kg |
| Oxalic Acid | 62 and 62 L |
| Demineralized Water | 744, 62, 248, 186, 248, |
| | 186, and 186 L |

Compound 10 (62 kg) wet cake is added to a reactor containing methylene chloride (1240 L) and stirred for 10 min. The mixture is heated to remove methylene chloride and water under azeotropic reflux. After methylene chloride (1550 L) is removed and no water remains in the distillate, the mixture is cooled to 25-30° C. An in-process sample is taken to determine water content (limit <0.1%).

Methylene chloride (186 L) is added to another reactor at 25-30° C. An in-process sample is taken to check for water content (limit ≦0.2% w/w). Aluminum chloride (81 kg) is added and the contents are stirred at 25-30° C. for 10-15 min. The mixture is cooled to 10-15° C. and octanethiol (78 kg) is added. The mixture is cooled to −20 to −10° C. The slurry of 10 in methylene chloride controlled at −20 to −7° C. is added to the stirred mixture that is temperature controlled at −15 to −10° C. for 20-30 min. The mixture is heated to 10-15° C. for 1.5-2.5 h. An in-process sample is taken to determine 10 content (limit ≦3.5%). The mixture is further cooled to −20 to −10° C. and then transferred to another reactor containing oxalic acid (62 kg), methylene chloride (186 L), and demineralized water (744 L) while maintaining the temperature below −3° C. to quench the reaction. The quenched material is slowly heated to 25-30° C. and maintained at this temperature for 12 h. Methylene chloride is distilled out at 25-30° C. under vacuum until the mixture volume is reduced to 1364 L. The mixture is centrifuged for 3 h and the wet cake is washed with demineralized water (62 L). The wet cake is added to a reactor containing oxalic acid (2.5 kg) and demineralized water (248 L) and the contents are stirred at 25-30° C. for 2 h to obtain a clear solution. The material is centrifuged for 1 h 15 min to 1 h 30 min and the wet cake is washed twice with demineralized water (186 L). The wet cake is added to a reactor containing demineralized water (248 L) at 25-30° C. and the contents are stirred for 2 h. The material is centrifuged for 1 h 30 min to 2 h and the wet cake is washed twice with demineralized water (186 L). The material is collected and placed into preweighed containers.

Expected yield of L-threo(N-phthaloyl-3-(3,4-dihydroxyphenyl)serine (11): 40-50 kg.

### Synthesis of L-threo (3,4-dihydroxyphenyl)serine

| Raw Material | Standard Quantity |
|---|---|
| L-threo(N-phthaloyl-3-(3,4- | 45 kg |
| dihydroxyphenyl)serine) (11) | |
| Sodium Bicarbonate | 17 kg |
| Hydroxylamine HCl | 14 kg |
| Celite Hyflo Super Cel | 90 kg |
| Methanol | 360, 450-675, 900, 90, 23, 23, and 23 L |
| Demineralized Water | 225 L |

Methanol (360 L) is added to a reactor and cooled to 20-25° C. Compound 11 (45 kg) is added to the reactor while stirring at 25-30° C. for 15-20 min. Demineralized water (225 L) and sodium bicarbonate (17 kg) are added to another reactor and cooled to 20-25° C. Hydroxylamine hydrochloride (14 kg) is added and mixed for 15-20 min at 20-25° C. to obtain a clear solution. The solution of 11 in methanol is transferred through a sparkler filter into a reactor. The hydroxylamine and sodium bicarbonate solution is added to the reactor while maintaining the temperature at 25-30° C. The reaction mixture is heated to 65-70° C. and refluxed for 16 h. An in-process sample is taken to determine 11 content (limit ≦3%). The material is cooled to 25-30° C. with mixing for 2 h.

The material is centrifuged for 1 h and the wet cake is washed three times with methanol (23 L). The wet cake is dried at 40-45° C. until water content is ≦1.0% w/w.

Expected yield of L-threo(3,4-dihydroxyphenyl)serine (12): 20-24 kg.

### Synthesis of L-threo(3,4-dihydroxyphenyl)serine hydrochloride

| Raw Material | Standard Quantity | |
|---|---|---|
| L-threo (3,4- | 22 | kg |
| dihydroxyphenyl)serine | | |
| Hydrochloric Acid | 13 | L |
| Isopropyl Alcohol | 132, 22, and 22 L | |
| Demineralized Water | 55 | L |

L-threo (3,4-dihydroxyphenyl)serine 12 (22 kg) material is added to a reactor containing demineralized water (55 L) and stirred for 15-30 min. The material is cooled to 20-25° C. and concentrated hydrochloric acid (13 L) is added to form L-threo(3,4-dihydroxyphenyl)serine hydrochloride) (13). The mixture is stirred for 30-45 min until a white thick suspension is observed. The mixture is stirred for an additional 2.0 h±15 min Isopropyl alcohol (132 L) is slowly added and the mixture is stirred for 5 hr±15 min. The mixture is cooled to 15-20° C. and stirred for 30-45 min.

The mixture is centrifuged for 30 min and the wet cake is washed twice with chilled isopropyl alcohol (22 L) at 15-20° C. The material is unloaded from the centrifuge and a sample is taken to check the individual impurity by HPLC (limit ≦0.05%) and purity by HPLC (limit ≧99.0%).

Reprocessing: If the individual impurity by HPLC does not meet the limit ≦0.05%, compound 13 is reprocessed by adding the material to a reactor containing demineralized water (28 L) and stirring for 15-30 min. Concentrated hydrochloric acid (3 L) is added at 20-25° C. and mixing is continued for 15-30 min. Continue mixing for 2 h±15 min at the same temperature. Isopropyl alcohol (74 L) is added over a period of 2-3 h at 25-30° C. Mixing is continued at 25-30° C. for 5 h±15 min followed by cooling to 15-20° C. and mixing for 30-45 min. The mixture is centrifuged for 30 min and washed twice with chilled isopropyl alcohol (22 L) and checked for the individual impurity by HPLC (limit ≦0.05%).

Expected yield of L-threo(3,4-dihydroxyphenyl)serine hydrochloride) (13): 19-20 kg

### Synthesis of L-threo(3,4-dihydroxyphenyl)serine

| Raw Material | Standard Quantity |
|---|---|
| L-threo(3,4-dihydroxyphenyl)serine | 19.5 kg |
| hydrochloride) (13) | |
| Hydrochloric Acid | 6 kg |
| Triethylamine | 14 kg |
| Methanol | 41, 58.5, 19.5, and 19.5 L |
| Activated Carbon | 1 kg |
| Celite Hyflo Super Cel | 0.2 kg |

Compound 13 (19.5 kg) is added to a reactor containing demineralized water (195 L) while stirring at 25-30° C. Concentrated hydrochloric acid (6 L) is added and mixed for 25-30 min. For complete dissolution, the contents can be mixed for another 15-20 min. Activated carbon (1 kg) and celite (0.2 kg) are added and mixed for 30-40 min. The mixture is filtered through a sparkler filter and the filter is washed with demineralized water (1×L). The filtrate is transferred to another reactor. A solution containing triethylamine (14 kg) and methanol (41 L) is slowly added to the reaction mass (reactor) while mixing. The pH of the filtrate is adjusted to 7.0-7.25 over a period of 3 h at 25-30° C. The contents are stirred for 20-30 min. An in-process sample is taken to confirm the pH is 7.0-7.25. The mixture is stirred for 3 h. The mixture is centrifuged for 1 h and the wet cake is washed twice with demineralized water (19.5 L). The wet cake is removed from the centrifuge and kept for a slurry wash. The wet cake is added to a reactor containing methanol (58.5 L) while stirring at 25-30° C. for 30-40 min. The material is centrifuged for 1 h and the wet cake is washed with methanol (19.5 L). The wet cake is unloaded from the centrifuge and retained for water washing.

The wet cake is added to a reactor containing demineralized water (39 L) while stirring for 30-40 min. The material is centrifuged for 10 min and the wet cake is washed twice with methanol (19.5 L). The wet cake is unloaded and a sample is taken to check the chloride content (<200 ppm). The wet cake is dried at 40-45° C. until the water content is ≦0.1% w/w. A sample is taken after 16 h of drying to confirm loss on drying is ≦0.1% w/w. The dry material is sieved through a sifter (400 micron) and packed. A sample is taken for quality control testing.

Expected yield of L-threo(3,4-dihydroxyphenyl)serine: 14-15 kg.

The droxidopa produced according to the present invention was comparable to that produced using the hydrazine process. To analyze the process, droxidopa was prepared using the known hydrazine method and using the method of the present invention. The products were analyzed, and the physical properties of the products produced were found to be comparable and essentially identical.

However, a few notable differences were observed. For example, the mean particle size (d50) of the droxidopa produced according to the hydrazine process was 172 μm, whereas the mean particle size (d50) of the droxidopa produced according to the present invention was 109 μm. Although the melting range temperatures, analyzed by hot stage microscopy, were largely comparable, the temperature at which all birefringence was lost varied slightly between the samples. In the sample prepared according to the known method, all birefringence was lost at 267.8° C., with noted complete decomposition, whereas this was observed at a lower temperature (240.4° C.) in the sample prepared according to the inventive method.

Polymorphism was investigated by crystallizing the product from various solvent. No evidence of polymorphism was observed by XRPD, IR spectroscopy, or thermal analysis of the batches crystallized from water in combination with other solvents (solvent systems indicated below in Table 2). Because droxidopa is not soluble in organic solvents other than as listed in Table 2, the investigation was conducted with crystals obtained from these solvents. The crystalline form of those samples was confirmed with XRPD, IR, and thermal analysis.

TABLE 2
Results of Polymorphism Studies of Droxidopa

| Crystallization solvent(s) | Crystalline form |
|---|---|
| Water | Identical to standard |
| Hot water | Identical to standard |
| Water/methanol 80/20 v/v | Identical to standard |
| Water/acetone 80/20 v/v | Identical to standard |
| Water/acetonitrile 80/20 v/v | Identical to standard |
| Hot methanol | Identical to standard |

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method for the preparation of droxidopa comprising the step of deprotecting a droxidopa precursor comprising an N-phthaloyl group in the absence of hydrazine to form droxidopa free of residual hydrazine.

2. The method of claim 1, wherein the deprotecting step comprises treating N-phthaloyl-3-(3,4-dihydroxyphenyl)serine with hydroxylamine.

3. The method of claim 1, wherein the droxidopa is enantomerically enriched for the L-threo isomer.

4. The method of claim 3, wherein the L-threo isomer is present at an optical purity of at least about 98%.

5. A method for the preparation of droxidopa, comprising the steps of:
a) converting piperonal to 2-amino-3-(benzo-1,3-diox-5-yl)-3-hydroxypropanoic acid 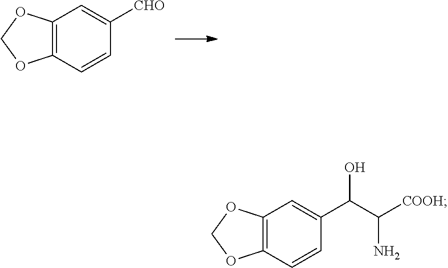 [SMILES: O=Cc1ccc2c(c1)OCO2 ; NC(C(=O)O)C(O)c1ccc2c(c1)OCO2]
b) protecting the free amine 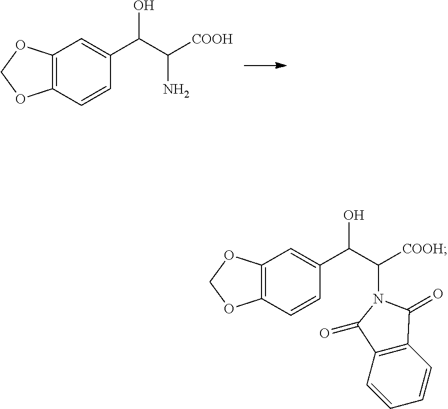 [SMILES: NC(C(=O)O)C(O)c1ccc2c(c1)OCO2 ; O=C(O)C(C(O)c1ccc2c(c1)OCO2)N1C(=O)c2ccccc2C1=O]
c) optical resolution and separation of the desired isomer 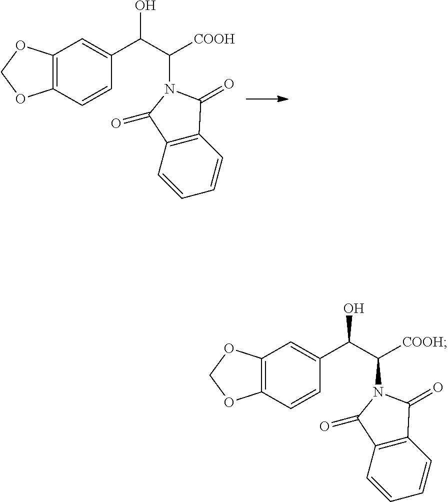 [SMILES: O=C(O)C(C(O)c1ccc2c(c1)OCO2)N1C(=O)c2ccccc2C1=O ; O=C(O)[C@H]([C@H](O)c1ccc2c(c1)OCO2)N1C(=O)c2ccccc2C1=O]
d) removal of the catechol protecting group 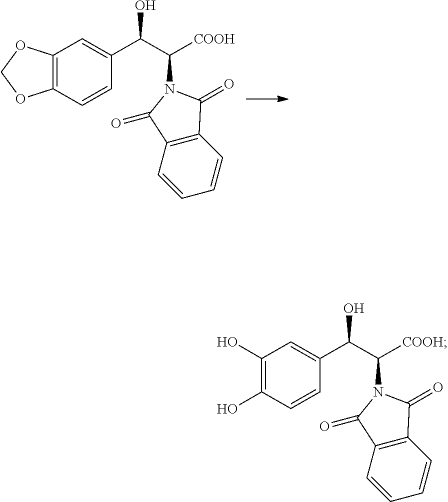 [SMILES: O=C(O)[C@H]([C@H](O)c1ccc2c(c1)OCO2)N1C(=O)c2ccccc2C1=O ; O=C(O)[C@H]([C@H](O)c1ccc(O)c(O)c1)N1C(=O)c2ccccc2C1=O]
and
e) removal of the phthaloyl protecting group

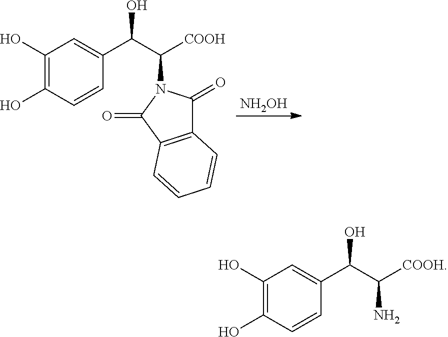

6. The method of claim 5, wherein step a) comprises adding glycine in the presence of a base.

7. The method of claim 6, wherein the base is sodium hydroxide or potassium hydride.

8. The method of claim 5, wherein step a) is conducted in an alcohol solvent.

9. The method of claim 8, wherein the alcohol solvent is methanol or ethanol.

10. The method of claim 5, wherein step b) comprises adding a phthaloylating agent selected from the group consisting of phthalic acid, phthaloyl chloride, phthalic anhydride, N-carbomethoxy pthalimide, N-carbethoxy pththalimide, monomethylphthalate, monoethyl phthalate, dimethyl phthalate, diethyl phthalate, and diphenyl pththalate.

11. The method of claim 10, wherein the method further comprises the step of reacting phthalimide with ClCOOMe to give N-carbomethoxy phthalimide.

12. The method of claim 11, wherein step b) comprises reacting the free amine with N-carbomethoxy phthalimide in the presence of Na₂CO₃.

13. The method of claim 5, wherein step c) comprises adding a chiral derivatizing agent selected from the group consisting of quinidine, quinine, strychnine, cinchonidine, cinchonine, ephedrine, norephedrine, 1-methylamine, dehydroabietylamine, R-2-amino-1,1-diphenyl-1-propanol, S-2-amino-1,1-diphenyl-1-propanol, and L-3-hydroxy-3(4-nitrophenyl)-2-amino-1-propanol.

14. The method of claim 13, further comprising adding an aqueous acidic solution to the product of step c) and extracting the desired isomer with an organic solvent.

15. The method of claim 13, wherein step c) comprises adding norephedrine in methanol to form an amine salt.

16. The method of claim 5, wherein step d) comprises adding a Lewis acid.

17. The method of claim 16, wherein the Lewis acid is selected from the group consisting of aluminum chloride, aluminum bromide, ferric chloride, stannic chloride, boron trichloride, and boron tribromide.

18. The method of claim 16, further comprising adding a mercaptan of 1-20 carbon atoms with the Lewis acid.

19. The method of claim 5, wherein step e) is conducted in a solvent selected from the group consisting of methanol, ethanol, water, and mixtures thereof.

20. The method of claim 1, wherein the droxidopa has an optical purity of greater than about 90%.

21. The method of claim 20, wherein the droxidopa has an optical purity of greater than about 95%.

22. The method of claim 21, wherein the droxidopa has an optical purity of greater than about 98%.

23. The method of claim 1, wherein the droxidopa comprises less than about 0.05% by weight hydrazine.

24. The method of claim 23, wherein the droxidopa comprises less than about 0.02% by weight hydrazine.

25. The method of claim 24, wherein the droxidopa comprises less than about 0.01% by weight hydrazine.

26. The method of claim 25, wherein the droxidopa comprises 0.0% by weight hydrazine.

27. L-threo-dihydroxyphenylserine, produced according to the method of claim 1.

28. A composition comprising droxidopa synthesized from an N-phthaloyl protected precursor, wherein the droxidopa is free of residual hydrazine.

29. The composition of claim 28, wherein the composition is a pharmaceutical composition comprising droxidopa and one or more pharmaceutically acceptable excipients.

30. The composition of claim 28, wherein the droxidopa is enantiomerically enriched for the L-threo isomer.

31. The composition of claim 30, wherein the L-threo isomer is present at an optical purity of at least about 98%.
